(19) United States
(12) Patent Application Publication
(10) Pub. No.: US 2011/0230913 A1
(43) Pub. Date: Sep. 22, 2011

(54) FACET JOINT PROSTHESIS

(75) Inventors: Stephan J. Duplessis, Calgary (CA); R. John Hurlbert, Calgary (CA); Lali Sekhon, Reno, NV (US)

(73) Assignee: KINETIC SPINE TECHNOLOGIES INC., Calgary (CA)

(21) Appl. No.: 13/087,600

(22) Filed: Apr. 15, 2011

Related U.S. Application Data
(63) Continuation of application No. PCT/CA2009/001428, filed on Oct. 14, 2009.
(60) Provisional application No. 61/106,067, filed on Oct. 16, 2008.

(51) Int. Cl. A61B 17/70

(52) U.S. Cl. 606/247

(57) ABSTRACT

A facet joint prosthesis comprises a pair of facet elements for engaging the superior and inferior articular processes of a facet joint. The facet elements are provided with one or more articulating surfaces for articulating movement there-between. The facet elements are also provided with a positive engagement means for preventing separation of the elements and/or to limit relative movement there-between within a pre-determined range. In one aspect, the invention provides a spacer for positioning between the facet elements and for distracting the facet joint. The spacer may also serve to form an artificial lateral mass between the superior and inferior articulating surfaces. The spacer may be provided with engagement means for positively engaging one or both of the facet elements.

### CROSS REFERENCE TO RELATED APPLICATIONS

This application is a Continuation of PCT application no. PCT/CA2009/001428, filed Oct. 14, 2009, which claims priority from U.S. Patent Application Ser. No. 61/106,067, filed Oct. 16, 2008. The entire contents of such prior applications are incorporated herein by reference.

### FIELD OF THE INVENTION

The present invention relates to artificial joint implants and, in particular, spinal implants. More specifically, the invention relates to spinal facet joint prostheses.

### BACKGROUND OF THE INVENTION

The spine is a complicated structure comprised of various anatomical components, which, while being extremely flexible, provides structure and stability for the body. A stable spine is important for preventing incapacitating pain, progressive deformity and neurological compromise. The spine is made up of vertebrae, each having a ventral body of a generally cylindrical shape. Opposed surfaces of adjacent vertebral bodies are connected together and separated by intervertebral discs (or “discs”), comprised of a fibrocartilaginous material. The vertebral bodies are also connected to each other by a complex arrangement of ligaments acting together to limit excessive movement and to provide stability.

Most vertebrae of the spine also include a pair of laminae, the anterior ends of which are each connected to a pedicle, which extend posteriorly from opposite sides of the vertebral body. The posterior ends of the laminae are connected to the posteriorly extending spinous process. The combination of the vertebral body, the pedicles, the laminae and the spinous process combine to form the spinal canal, and the vertebral foramen. Each lamina includes superior and inferior articular processes on opposite lateral sides thereof. Each superior process engages the opposing inferior process of the adjacent vertebrae located immediately superior thereto. The opposed superior and inferior processes form a facet joint. Thus, each pair of vertebrae is connected by two facet joints. Facet joints are diarthroidal joints, wherein opposed process surfaces are covered with cartilage. Such a structure allows sliding articulation between opposed superior and inferior processes forming the facet joint. The facet joints also assist in stabilizing the spine by supporting axial, torsional and shear loads applied thereto. Each facet joint is positioned at each level to provide the needed limits to motion, especially to rotation and to prevent forward slipping (spondylolisthesis) of a vertebra over the one below. For various reasons, such as disease, injury, damage, etc., facet joints become degenerated often resulting in severe pain to the patient. In such cases, one or both the processes forming the facet joint may be removed to alleviate the condition. Such dismantling of the facet joint often leads to destabilization of that region of the spine. For this reason, various artificial facet joints have been proposed. Examples of prior art facet joint prostheses are provided in the following U.S. Pat. No. and application Ser. Nos.: Re. 36,758; 7,041,136; 2005/0177240; 2005/0131538; 2007/0055373; 2007/0123863; 2008/0097612.

Although the known facet joint prostheses solve a number of associated problems, none have yet been shown to effectively and simply mimic the articulation of natural facet joints. In many cases, the known prostheses are complex in nature and, in most cases, serve to simply fuse the joint (by permanently joining opposed processes), which often results in stresses placed on adjacent vertebral structures. In other cases, such as taught in Re. 36,758, the known prostheses simply cover the processes with a cap or covering element. In these cases, although the surfaces of the processes are protected from further damage, the motion limiting nature of the facet joint is removed thereby resulting in destabilization of the spine.

Thus, there exists a need for an effective artificial facet joint that overcomes at least some of the deficiencies associated with known prostheses.

### SUMMARY OF THE INVENTION

In one aspect, the present invention provides a facet joint prosthesis that provides a desired degree of articulation.

In another aspect, the invention provides a facet joint prosthesis being adapted to limit articulation to a constrained region and to prevent separation of the joint. Thus, in one aspect, the invention provides a facet joint prosthesis system comprising:

a superior facet element adapted to engage an inferior process of a first vertebra;

an inferior facet element adapted to engage a superior process of a second vertebra;

the first and second vertebrae being adjacent to each other in a spine;

the superior facet element having at least one first articulation surface, the inferior facet element having at least one second articulation surface, wherein, when in use, each first articulation surface articulates against each of the respective second articulation surfaces; and,

a first engagement means provided on the superior facet element for engaging a second engagement means provided on the inferior element, wherein, when in use, the first and second engagement means inhibit separation of the superior and inferior facet elements. In another aspect, the facet elements of the prosthesis system are provided with a positive engagement means to prevent separation of the facet elements and/or to limit relative movement there-between to a pre-determined range.

In another aspect, the prosthesis system further includes a spacer for positioning between the facet elements and for maintaining the facet joint in a desired distracted position. In a further aspect, the spacer includes one or more engagement means to positively engage one or both of the facet elements.

### BRIEF DESCRIPTION OF THE DRAWINGS

These and other features of aspects of the invention will become more apparent in the following detailed description in which reference is made to the appended drawings wherein:

FIG. 1 is perspective view of a spinal segment illustrating a pair of vertebrae and the two facet joints there-between.

FIG. 2 is a posterior view of a spinal segment illustrating a pair of vertebrae and the two facet joints there-between.

FIG. 3 is a posterior perspective view of the facet joint of the invention according to one aspect.

FIG. 4 is an anterior view of the joint of FIG. 3.

FIG. 5 is a posterior view of the superior articulating element of FIG. 3.

FIG. 6 is a posterior perspective view of the element of FIG. 5.

FIG. 7 is an anterior view of the element of FIG. 5.

FIG. 8 is a posterior view of the inferior articulating element of FIG. 3.

FIG. 9 is a posterior perspective view of the element of FIG. 8.

FIG. 10 is an anterior perspective view of the element of FIG. 8.

FIG. 11 is a posterior view of a spacer according to an aspect of the invention.

FIG. 12 is a perspective view of the spacer of FIG. 11.

FIG. 13 is an anterior view of the spacer of FIG. 11.

FIG. 14 is an exploded posterior perspective view of the facet joint of FIG. 3 with the spacer of FIG. 11.

FIG. 15 is a posterior perspective view of the facet joint of FIG. 3 with the spacer of FIG. 11.

FIG. 16 is an anterior perspective view of the joint of FIG. 15.

### DETAILED DESCRIPTION OF EMBODIMENTS OF THE INVENTION

In the following description, the terms “superior”, “inferior”, “anterior”, “posterior” and “lateral” will be used. These terms are meant to describe the orientation of vertebrae or the implants of the invention when positioned in the spine and when the spine is in the upright position. Thus, “superior” refers to a top portion and “posterior” refers to that portion of the implant (or other spinal components) facing the rear of the patient's body when the spine is in the upright position. Similarly, the term “inferior” will be used to refer to the bottom portions of the implant while “anterior” will be used to refer to those portions that face the front of the patient's body when the spine is in the upright position.

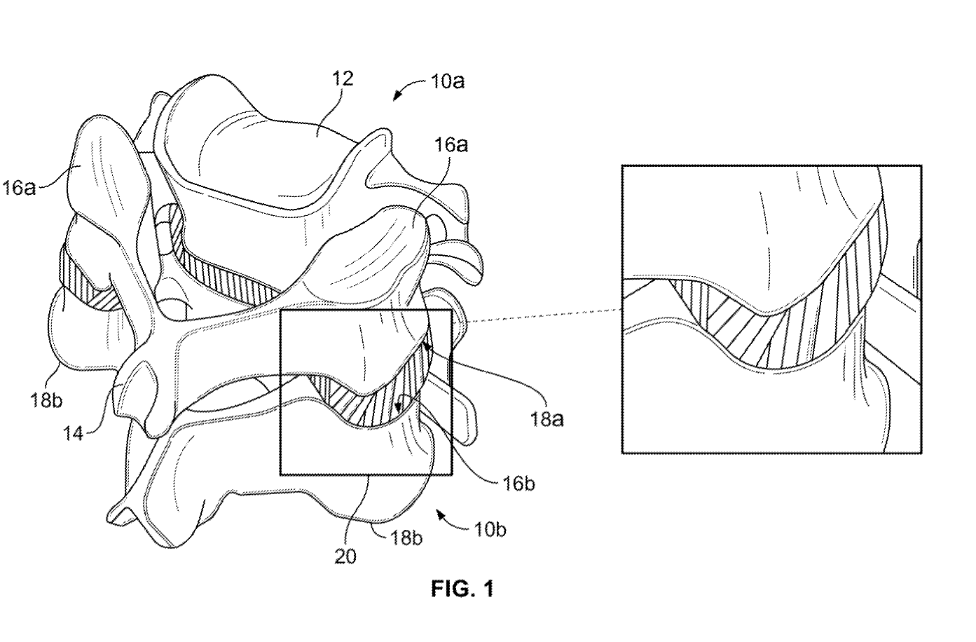

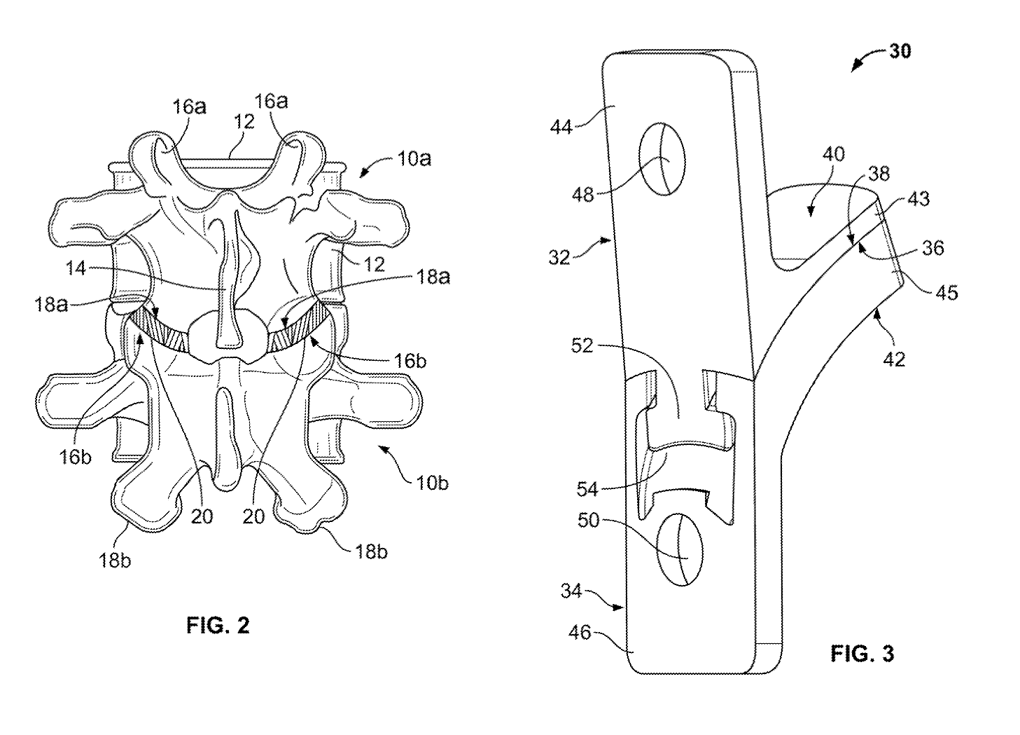

As shown in FIGS. 1 and 2, a spinal segment, as the term is used herein, comprises superior and inferior vertebrae 10a and 10b, respectively. Each vertebra comprises a vertebral body 12 and a spinous process 14. As indicated above, each vertebra also includes a pair of superior articular processes 16a, b and a pair of inferior articular processes 18a, b. As shown in FIGS. 1 and 2, a facet joint 20 between a pair of adjacent vertebrae, 10a and 10b, comprises a superior process 16b of the inferior vertebra 10b engaged with the inferior process 18a of the superior vertebra 10a. As shown, the facet joint includes a cartilaginous material positioned between the opposed processes so as to provide lubrication for the joint 20.

As will be known to persons skilled in the art, the range of motion offered by a facet joint depends upon the position of the vertebrae along the length of the spine. That is, the range of motion offered by facet joints for cervical vertebrae is different from that offered for thoracic or lumbar vertebrae. There may also be a variation between facet joints in the same region of the spine. These variations are the result of varying orientations of the facet joint at the various levels of the spine. For example, facet joints may extend from being parallel to the transverse plane, such as for the C1 and C2 vertebrae, to being orthogonal to the transverse plane, such as for lumbar vertebrae. In the result, the facet joints of cervical vertebrae are capable of flexion, extension, lateral flexion and rotation whereas the facet joints of lumbar vertebrae are capable of flexion, extension and lateral flexion but only limited rotation.

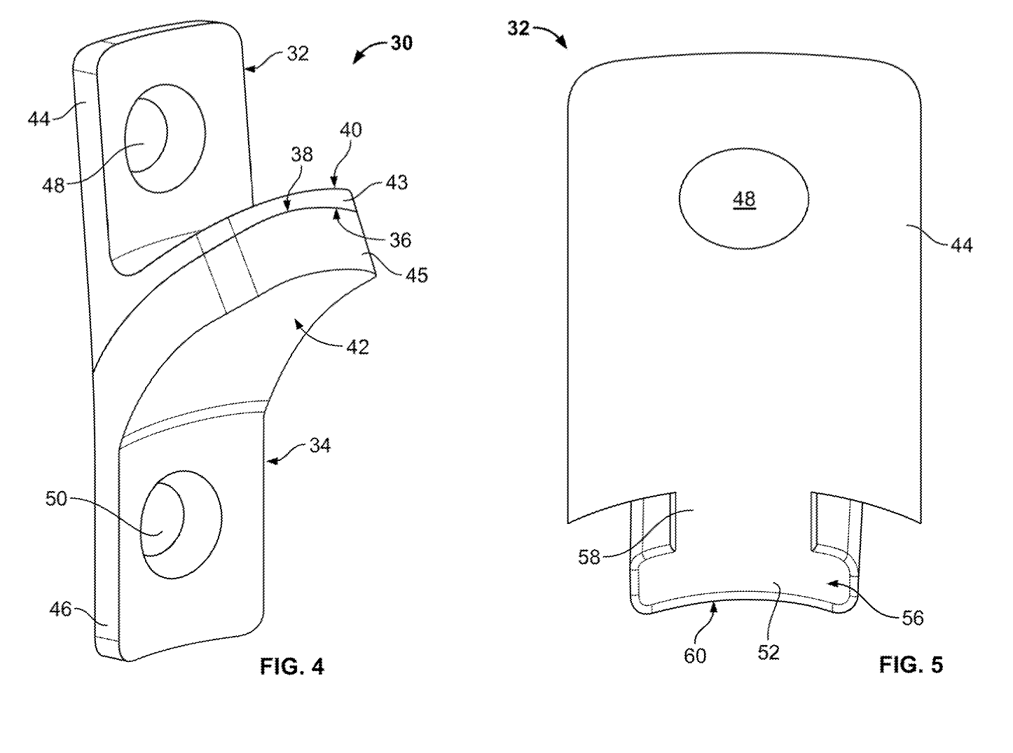

FIGS. 3 and 4 illustrate one aspect of the present invention. As shown, the illustrated aspect of the facet joint prosthesis 30 is comprised of two articulating elements, namely, a superior facet element 32 and an inferior facet element 34. The superior element 32 is adapted to be secured to the inferior articular process of the superior vertebra while the inferior element 34 is adapted to be secured to the superior articular process of the inferior vertebra of a spinal segment. The inferior surface 36 of the superior element 32 and the superior surface 38 of the inferior element 34 are formed so as to allow articulation there-between. In the embodiment illustrated, the surface 36 is formed with a generally concave shape and the surface 38 is formed with a generally convex shape. In this way, the surfaces 36 and 38 are allowed to smoothly articulate as the processes forming the facet joint are moved relative to each other. In one aspect as shown in the figures, the surfaces 36 and 38 may optionally be curved in two planes thereby allowing the surfaces to articulate in a variety of directions. It will be understood that the amount or degree of articulation may be limited by varying the convex or concave structures of the surfaces 36 and 38.

Each of the elements 32 and 34 further include bone engaging surfaces. As shown, the superior element 32 includes a superior surface 40 for contacting and engaging the inferior process of the superior vertebra. Similarly, the inferior element 34 includes an inferior surface 42 for contacting and engaging the superior process of the inferior vertebra. As will be understood, the surfaces 40 and 42, including any other bone contacting surfaces of the invention, may be optionally provided with any means for enhancing bone contact. For example, such surfaces may be provided with a textured or roughened surface for preventing movement between the surface and the bone to which it is engaged. Alternatively, such surfaces may be provided with keels or spikes etc. for engaging and gripping adjacent bone. Further, the surfaces may optionally be provided with bone in-growth promoting surface structures (i.e. apertures, pores etc.) and/or may be coated with a bone in-growth promoting agent. Various other means of promoting engagement of the elements to the adjacent bone surfaces will be known to persons skilled in the art. The present invention may utilize one or more such means or, alternatively, no means at all.

In one aspect of the invention, the bone engaging surfaces of the elements 32 and 34 may be adapted to enhance bone contact and fixation of the prosthesis by being provided with outwardly extending teeth. In a further aspect, the teeth may be provided in an angulated format similar to a saw tooth arrangement. In such manner, movement or displacement of the prosthesis is prevented once it is implanted in position against bone surfaces. That is, the teeth may be provided on the bone engaging surfaces of the prosthesis in a direction that is counter to any displacement or dislodgment forces that may be experienced during use of the prosthesis. The teeth described above can alternatively be provided in any orientation with respect to the surfaces from which the extend. For example, in one aspect, the teeth may project generally perpendicularly, that is, without any angular direction. Alternatively, the teeth may be provided in different directions to counter multi-directional displacement forces. The teeth may also be arranged in rows each row having the same or different directional orientation.

In one aspect, the articulating elements 32 and 34 are provided with a generally “L” shaped cross section. As shown in FIGS. 3 and 4, the superior element 32 includes a generally axially extending flange 43 and a generally longitudinally extending flange 44. The flanges 43 and 44 of the superior element 32 are arranged to form generally acute angle there-between so as to engage the inferior process of the respective vertebra. In a similar manner, the inferior element 34 includes a generally axially extending flange 45 and a generally longitudinally extending flange 46. The flanges 45 and 46 of the inferior element 34 are arranged to form a generally obtuse angle there-between so as to facilitate engagement with the respective superior process. As shown the longitudinal flange 44 of the superior element 32 extends in the superior (i.e. upward) direction and is adapted to contact the posterior surface of the inferior process of the superior vertebra. Similarly, the flange 46 of the inferior element 34 extends in the inferior direction and is adapted to contact the posterior surface of the superior process of the inferior vertebra. As described above, the surfaces of the flanges that contact bone may be optionally provided with a bone engaging or bone in-growth promoting surface or treatment. In one aspect of the invention as shown in the figures, the flanges 44 and 46 optionally include apertures 48 and 50, respectively, for receiving bone screws there-through. As will be understood, such screws (not shown) would serve to anchor the elements to the respective bone structure. Bone screws of this type are commonly known in the art. It will be understood that any other securing means may be used to anchor the elements to the neighbouring bone structures, where such anchoring is desired. For example, instead of the screws mentioned above, the elements 32 and 34 may be secured or anchored to bone by means of hooks, straps, staples, adhesives and other such means as will be known to persons skilled in the art.

As shown in FIG. 3, the surfaces 36 and 38 are preferably provided with cooperating structures for limiting the range of motion between the elements 32 and 34. In one aspect, as illustrated, this motion limiting structure or arrangement comprises a tongue and groove assembly wherein the superior and inferior articulating elements are engaged and prevented from separating axially. In another aspect, this tongue and groove arrangement is provided with one or more articulating surfaces to allow the elements 32 and 34 to move in varying directions in a controlled manner. In the aspect of the invention illustrated in FIG. 3, the superior articulating element 32 includes a tongue structure 52 while the inferior element includes a cooperating groove structure 54. It will be understood that this arrangement may be reversed. Both the tongue 52 and groove 54 are adapted to conform to the contour of the respective inferior surface 36 and superior surface 38. That is, the tongue 52 is formed is provided as a generally concave form while the groove is provided as a cooperating generally convex form.

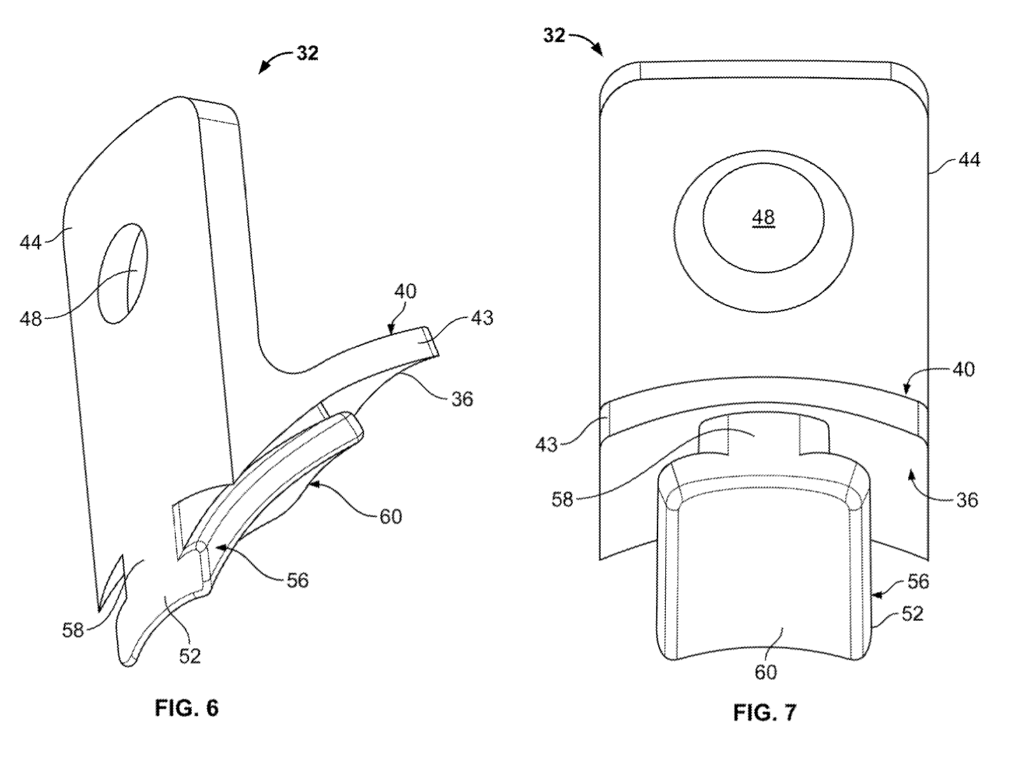

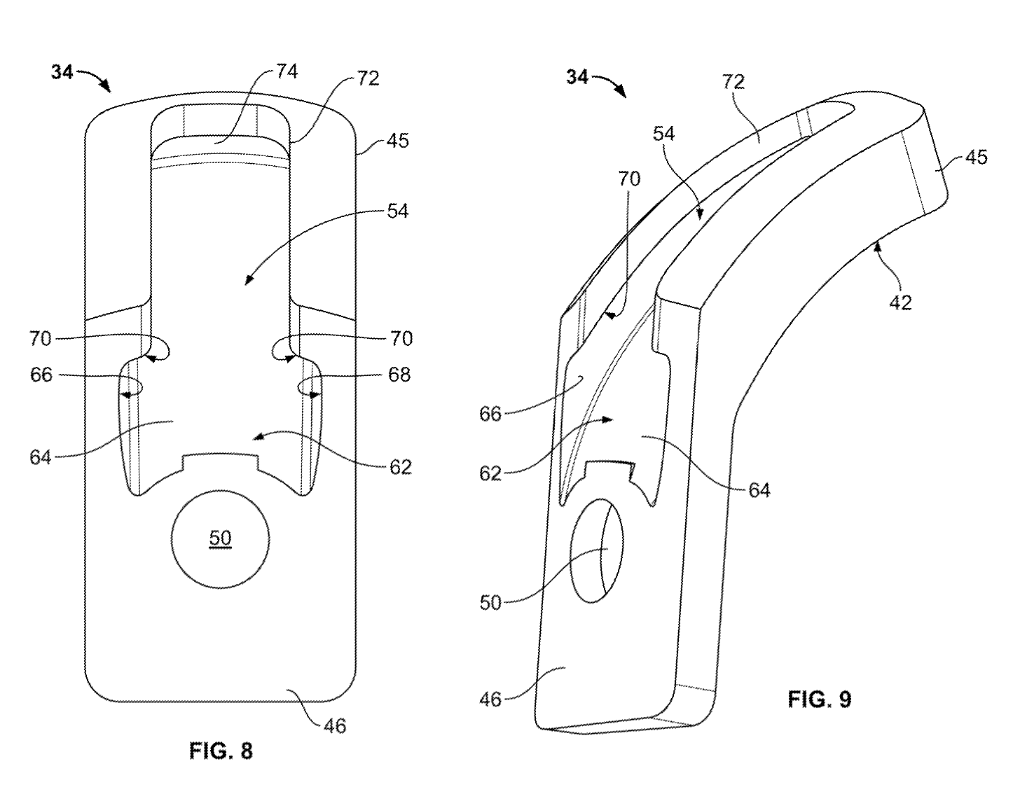

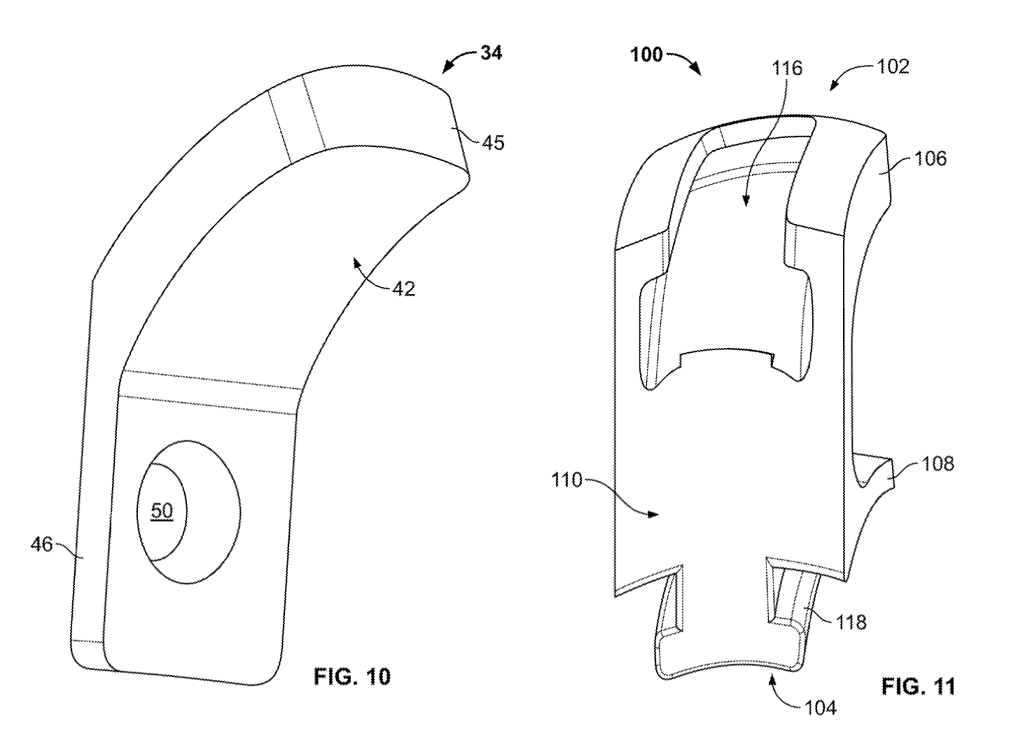

The above mentioned tongue and groove arrangement between the articulating elements 32 and 34 is further illustrated in FIGS. 5 to 10, where common reference numerals are used to identify common features. FIGS. 5 to 7 illustrate the superior element 32 in isolation while FIGS. 8 to 10 illustrate the inferior element 34 in isolation.

As shown in FIGS. 5 to 7, the tongue 52 of the superior articulating element 32 is provided with a generally inverted “T” shape in end cross section, having a wide articulating portion 56 connected to the axial flange 43 by means of a thin stem 58. The articulating portion 56 comprises a generally elongate structure extending over the all or a portion of the length of the axial flange 43. The inferior surface 60 of the tongue 52 is convex in both the axial and lateral directions.

FIGS. 8 to 10 illustrate the inferior articulating element 34 and the “groove” structure 54 provided therewith. As shown, the groove 54 is adapted to slidably receive the tongue 52. To accommodate the tongue 52, the groove 54 is provided with an elongate articulating recess 62 to receive the articulating portion 56 of the tongue. The slot 62 is provided with a convex superior surface 64 that is shaped to cooperate with the inferior surface 60 of the tongue. That is, the inferior surface 60 of the tongue, when engaged within the recess 62 is capable of articulating movement similar to a ball and socket type of connection. As will be understood, this arrangement allows the superior and inferior elements 32 and 34 to be moved relative to each other in an articulating manner.

The tongue 52 and groove 54 assembly is also provided with various motion inhibiting structures so as to limit the articulation between the elements 32 and 34 to within a pre-determined range. In this regard, the recess 62 of the groove 54 is provide with side walls 66 and 68 extending along the flange 45 and a superior wall 70 with a generally central slot 72. The slot 72 is adapted to receive the stem 58 of the tongue 52 of the superior element 32. In such arrangement, when the elements 32 and 34 are engaged, the articulating portion 56 of the tongue 52 is constrained within the recess 62 and prevented from being laterally or axially separated there-from. Similarly, the recess 62 is provided with an end wall 74 positioned anteriorly of the element 34 to restrict longitudinal movement of the tongue 52 within the groove 54. The posterior end of the groove 54 is provided with an opening through which the tongue 52 is inserted when assembling the present facet joint.

The relative dimensions of the tongue 52 and groove 54 may be adjusted to allow a desired range of movement there-between. For example, in order to allow lateral bending of the joint, the groove 54 may be provided with side walls 66 and 68 that are wider than the articulating portion 60 of the tongue 52. In this manner, once the elements 32 and 34 are engaged (i.e. when the tongue 52 is provided within the groove 54), the superior element 32 and inferior element 34 are allowed a degree of relative lateral motion. As will be understood, in this arrangement, the joint 30, once implanted, will allow the spinal segment a range of lateral bending movement. Similarly, the opening provided on the groove 54 allows the tongue 52 to be moved posteriorly there-through. Such an arrangement provides the joint 30 with a degree of extension movement as the superior element 32 is moved posteriorly over the inferior element 34. In addition, the length of the groove 54 may be adjusted to accommodate a degree of anterior movement of the tongue 52 there-within by positioning the end wall 74 more anteriorly. In this arrangement, the joint 30 is provided with a degree of flexion movement as the superior element 32 is moved anteriorly with respect to the inferior element 34. Thus, the walls 66, 68 and 74 act as “hard stops” to restrict movement of the tongue 52 within the groove 54.

As will be understood in the above description and the accompanying figures, the joint 30 of the present invention is provided with a variety of articulating surfaces between the superior and inferior elements 32 and 34. For example, as described above, the tongue 52 and groove 54 engage each other with cooperating articulating surfaces, 60 and 62. Further, it will be understood that the height of the groove 54 may be adjusted so as to have the superior wall 70 rest or bear upon the superior surface of the articulating portion 60 of the tongue 52. In addition, the inferior surface 36 of the superior element 32 and the superior surface 38 of the inferior element 34 also bear against each other. These various contact surfaces may be, as indicated above, oppositely curved so as to provide a complementary articulating engagement there-between. As a result of one or more of these pairs of contacting surfaces, and one or more of the motion limiting means described above, movement between the elements 32 and 34 may be allowed in a controlled manner. Further, by sizing the tongue and groove accordingly, the joint 30 may be allowed to undergo one or more of flexion, extension, rotation, or any combination of these motions. Such movement can, therefore, be tailored to meet the required physiological criteria depending upon the location of the joint along the spine. For example, a joint 30 placed in one region of the spine may be provided with more degrees of motion freedom than another. The desired amount of relative movement or restriction will be understood by persons skilled in the art.

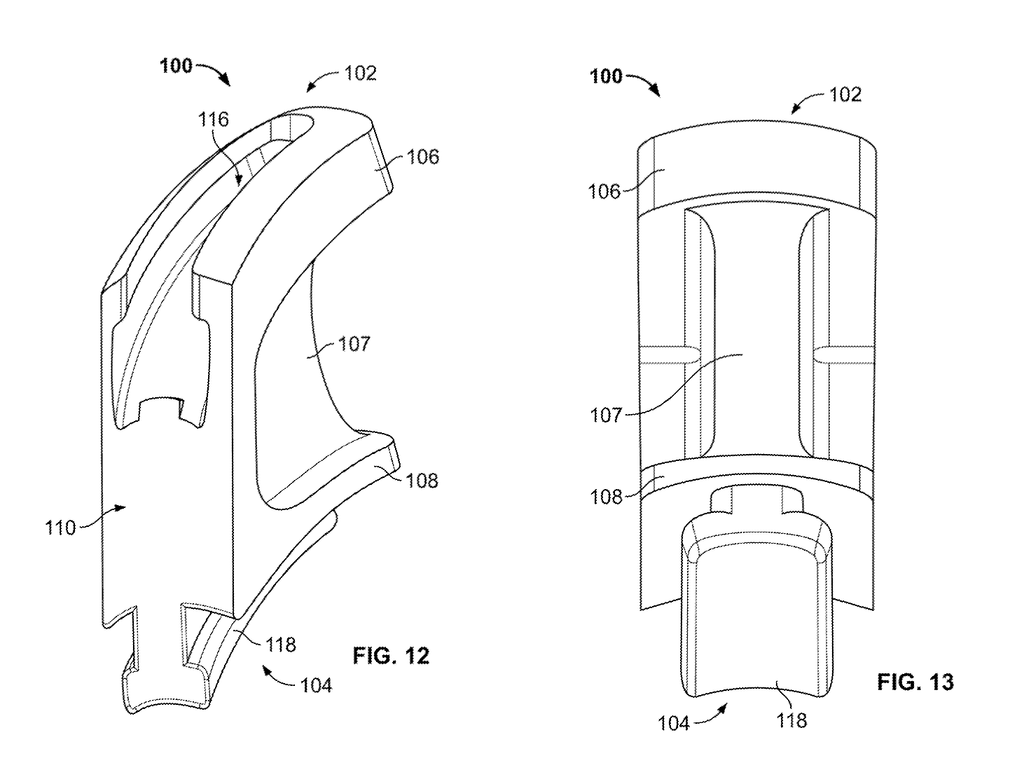

In certain cases, distraction of a facet joint may be needed to correct the alignment of adjacent processes. Such distraction may be needed to restore kyphosis or otherwise restore the curvature of the spinal segment. To accommodate this, the artificial joint of the invention may include a spacer or other type of spacing means to separate the superior and inferior elements 32 and 34 by any given amount. One embodiment of such a spacer is illustrated in FIGS. 11 to 13. As shown, the spacer 100 includes a superior end 102 and an inferior end 104. The spacer 100 is adapted to be provided between the elements 32 and 34 described above. Thus, in one aspect, the superior end 102 of the spacer is adapted to engage the superior element 32 and the inferior end 104 of the spacer is adapted to engage the inferior element 34. To facilitate the engagement of the spacer 100 with the elements 32 and 34, the spacer may be provided, in one aspect, with a means of receiving the tongue 52 and/or the groove 54 described above. The spacer also acts as an artificial lateral mass, that may serve to promote stability or allow for potential anchoring of other fixations to this structure.

In one embodiment of the invention, the spacer 100 comprises a generally “C” shaped structure having a superior flange 106 and an inferior flange 108. The flanges 106 and 108 are separated by a supporting or stiffening rib 107 extending there-between. Both flanges 104 and 106 extend anteriorly from the posterior end 110. The superior surface 112 of the superior flange 106 is provided with a convex shape for engaging the concave inferior surface 36 of the superior element 32. Similarly, the inferior surface 114 of the inferior flange 108 is provided with a concave shape for engaging the convex superior surface 38 of the inferior element 34. Thus, as will be understood, each of the elements 32 and 34 engage the spacer 100 in an articulating arrangement similar to the arrangement of the elements themselves as described above.

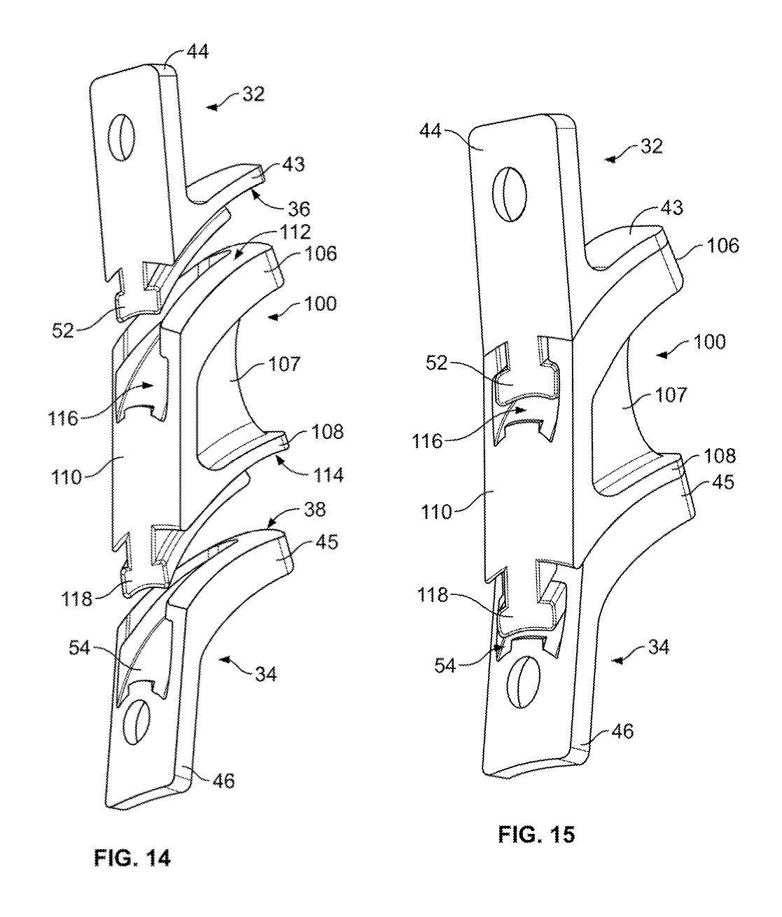

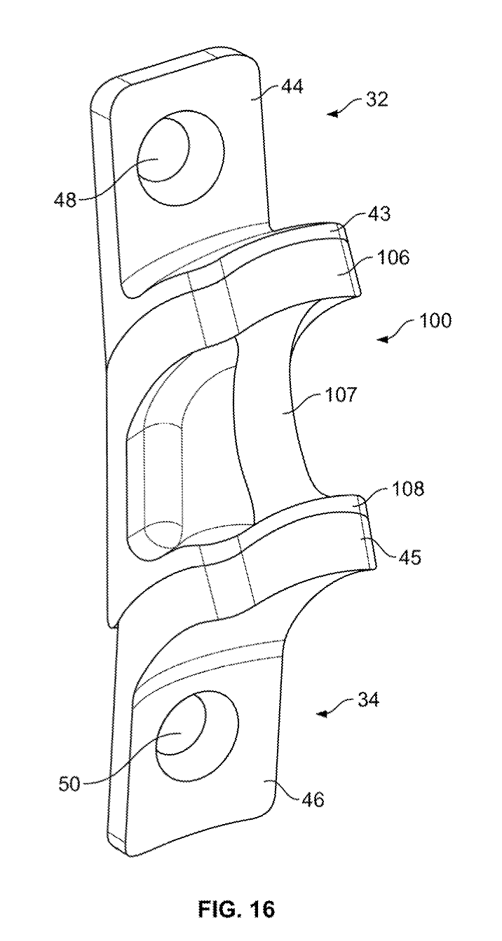

In addition, the superior end 102 of the spacer 100 may be provided with a groove 116 that is similarly shaped as the groove 54 provided in the inferior element 34 described above and is designed to function in similar manner. In addition, or alternatively, the inferior end 104 of the spacer 100 may be provided with a tongue 118, which is similar in shape and function to the tongue 52 provided on the superior element 32 as described above. In this way, and as illustrated in FIGS. 14 to 16, the groove 116 of the superior end 102 of the spacer is adapted to engage the tongue 52 of the superior element 32. Similarly, the tongue 118 of the inferior end 104 of the spacer is adapted to be received within the groove 54 of the inferior element 34. In addition, the surfaces of the superior end 102 and inferior end 104 are preferably adapted to engage and articulate with the adjacent surfaces 36 and 38, respectively, of the superior and inferior elements 32 and 34, when the components are combined. As will be understood, the relative movements between the elements 32 and 34 and the spacer 100 would, therefore, be similar to the relative movement between the elements 32 and 34 themselves if no spacer was provided.

The various components of the invention can be made from the same or different material. A variety of such materials as will be known to persons skilled in the art. For example, the components may be manufactured from metals (such as stainless steel, titanium, titanium alloys, nickel-titanium alloys, such as Nitinol™, cobalt-chrome alloys, etc.), porcelain, ceramics, and plastic and/or thermoplastic polymers (such as PEEK™), or any combination thereof. In addition, the flanges of the elements and spacers discussed above may be provided with one or more layers or coatings comprising a material that is different from the flange itself.

Although the present invention has been defined with respect to a facet joint prosthesis, it will be understood that the components thereof can be used for any other joint prosthesis in other parts of the body.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

What we claim is:

1. A facet joint prosthesis system comprising:
a superior facet element adapted to engage an inferior process of a first vertebra;
an inferior facet element adapted to engage a superior process of a second vertebra;
the first and second vertebrae being adjacent to each other in a spine;
the superior facet element having at least one first articulation surface, the inferior facet element having at least one second articulation surface, wherein, when in use, each first articulation surface articulates against each of the respective second articulation surfaces; and,
a first engagement means provided on the superior facet element for engaging a second engagement means provided on the inferior element, wherein, when in use, the first and second engagement means inhibit separation of the superior and inferior facet elements.

2. The prosthesis system of claim 1 wherein the superior and inferior facet elements are provided with a respective plurality of articulation surfaces.

3. The prosthesis system of claim 1 wherein the first and second engagement means cooperate to allow movement between the superior and inferior facet elements in at least one plane.

4. The prosthesis system of claim 1 wherein the first and second engagement means cooperate to allow at least one of flexion, extension and rotation movements between the superior and inferior facet elements or any combination of such movements.

5. The prosthesis of claim 4 wherein the first and second engagement means cooperate to restrict said movements to within a pre-determined range.

6. The prosthesis system according to claim 1 wherein one of the first and second engagement means comprises a tongue and the other of the first and second engagement means comprises a groove adapted to receive said tongue.

7. The prosthesis system of claim 6 wherein the tongue is provided with a widened distal end and wherein the groove is provided with a complementary shape to positively engage the tongue and to prevent separation thereof once engaged within the groove.

8. The prosthesis system of claim 1 wherein one of the first and second articulation surfaces are convexly shaped and the other of said first and second articulation surfaces are concavely shaped and adapted to receive said convexly shaped surface.

9. The prosthesis system of claim 1 wherein at least one of the superior and inferior facet elements include a means for securing said element to an adjacent bone structure.

10. The prosthesis system of claim 1 further comprising a spacer element for positioning between the superior and inferior facet elements.

11. The prosthesis system of claim 10 wherein the spacer element includes a superior surface for articulating against the first articulation surface of the superior facet element and an inferior surface for articulating against the second articulation surface of the inferior facet element.

12. The prosthesis system of claim 11 wherein the superior surface of the spacer includes a third engagement means for engaging the first engagement means of the superior facet element.

13. The prosthesis system of claim 11 wherein the inferior surface of the spacer includes a fourth engagement means for engaging the second engagement means of the inferior facet element.
